(19) United States
(12) Patent Application Publication
(10) Pub. No.: US 2009/0088393 A1
(43) Pub. Date: Apr. 2, 2009

(54) METHODS AND FORMULATIONS FOR CONVERTING INTRAVENOUS AND INJECTABLE DRUGS INTO ORAL DOSAGE FORMS

(75) Inventor: CURTIS A. SPILBURG, Chesterfield, MO (US)

Correspondence Address: MCKEE, VOORHEES & SEASE, P.L.C., 801 GRAND AVENUE, SUITE 3200, DES MOINES, IA, 50309-2721, US

(73) Assignee: ZOMANEX, LLC, CHESTERFIELD, MO (US)

(21) Appl. No.: 11/864,113

(22) Filed: Sep. 28, 2007

(51) Int. Cl. A61K 31/704; A61K 31/728; A61K 31/439; A61K 31/4375

(52) U.S. Cl. 514/27; 514/54; 514/283; 514/34

(57) ABSTRACT

Oral dosage compositions for drugs normally given intravenously such as Paclitaxel, containing a plant sterol to enhance solubility and a small intestine efflux inhibitor to prevent P-glycoprotein from being a barrier to absorption.

### FIELD OF THE INVENTION

This invention relates to a general method for enhancing the bioavailability of hydrophobic drug active compounds, using naturally-occurring formulation ingredients that are present in the diet. Specifically, this invention is especially useful as a general formulation method for the delivery of drugs in liquid or dry form for oral dosing that heretofore have been administered intravenously or by injection.

### BACKGROUND OF THE INVENTION

Oral drug delivery, the preferred method of administration for most people, remains a subject of intense pharmaceutical and biochemical investigation since the mechanism(s) of drug absorption in the small intestine is largely unknown. It is generally believed that two processes control the amount of drug that is absorbed. First, a high concentration of the active substance at the intestinal membrane surface will enhance cellular absorption (Fick's Law) and, since cells function in an aqueous environment, enhancing the water solubility of a drug increases its concentration at the locus of absorption. However, even though greater water solubility may be expected to enhance the bioavailability of drugs, this is frequently not the case due to a second, competing process that affects the overall absorption process. The absorptive cell membrane is composed mainly of lipids that prevent the passage of hydrophilic water-soluble compounds, but which are highly permeable to lipid soluble substances. Therefore, the design of bioavailable drugs must balance these two opposing forces. On the one hand, a drug that is very hydrophilic may have a high concentration at the cell surface but it may be impermeable to the lipid membrane. On the other hand, a hydrophobic drug that may easily “dissolve” in the membrane lipids may be virtually insoluble in water producing a very low concentration of the active substance at the cell surface. The inherent conflict, for effective oral dosing thus becomes apparent.

The intestinal plasma membrane lines the lumen of the upper gut and is the first absorptive surface to be permeated by most nutrients, foodstuffs and oral dosed drugs. As part of the digestive process, the apical side of the cell is exposed to a complex milieu consisting of pancreatic enzymes, bile and partially digested food from the stomach. Drug absorption does not occur in isolation. Since most drugs are lipophilic, their absorption takes place along with or in competition with that for other lipophilic molecules, such as cholesterol, fat-soluble vitamins, oils and fatty acids. The small intestine is densely covered with villi and microvilli, which greatly enhance the area available for absorption (250 m²), favoring the uptake of even poorly soluble substances. Moreover, the cell surface is also covered with heparin, a negatively charged polysaccharide that tightly binds lipolytic enzymes, such as cholesterol esterase and triglyceride lipase, providing a locus of hydrolytic activity virtually contiguous with the absorptive surface (Bosner M S, et al., Proc Nat'l Acad Sci 85: 7438-7442, 1989). This tight binding interaction ensures a high level of lipolytic activity even when the pancreas is not secreting enzymes.

The combination of lipolytic enzymes, bile components and a large intestinal absorption surface provides an environment in which virtually all food is absorbed (Armand M et al., Am J Physiol 271: G172-G183, 1996). While the above-mentioned processes are extremely efficient, the same is not true for certain chemically complex lipids, such as cholesterol, plant sterols, fat soluble vitamins, naturally occurring dietary nutrients, xenobiotics and drugs. Over the past twenty years, much progress has been made in delineating the biochemical processes that are used for the net absorption of these types of compounds, and a central feature of this new understanding is the identification, isolation and dynamic interplay of individual intestinal proteins in the overall absorption process. For drug uptake, the ATP-binding cassette transporter P-glycoprotein (P-gp) plays a pivotal role in modifying the absorption process. Located in high concentration on the villus tip of the apical surface of the brush border membrane, P-gp can serve as a barrier for the intestinal absorption of numerous drug substrates by pumping absorbed drug back into the intestinal lumen (Pang K S, Drug Metab Disp 31: 1507-1519, 2005). Thus, increasing the dispersibility of a hydrophobic drug may be thwarted if it is also a substrate of the efflux protein P-gp.

Aqueous dispersibility and susceptibility to small intestinal cell efflux transporters are central problems that therefore must be overcome in order to prepare an oral dosage form for hydrophobic drugs and especially xenobiotics. If these problems cannot be solved then the drug must be given by an alternative methodology, typically intravenously or by injection. These absorption problems are exemplified by (but not limited to) xenobiotics, naturally occurring plant- or marine-derived compounds that have interesting pharmacological properties. Taxanes, camptothecins, anthrocyclines, epipodophyllotoxins, and vinca alkaloids are potent anti-cancer agents that are difficult to formulate in oral dosage forms. To circumvent these delivery problems the oral solid delivery approach is frequently abandoned in favor of an emulsion-based, liquid intravenous strategy. For example, paclitaxel, a potent anti-cancer agent isolated from yew needles, is currently administered intravenously as a dispersion in Cremophor EL, an ethanol blend of castor oil, to create an emulsified paclitaxel dispersion. While this delivery strategy is effective, there are a number of drawbacks that may limit the usefulness of the drug, both from a patient and a biochemical perspective. For example, the intravenous administration occurs in a clinical setting that causes a major disruption in daily activities. This is further complicated by severe hypersensitivity reactions that are the by-product of the Cremophor emulsification system (van Zuylen, L et al., Invest. New Drugs, 2001, 19: 125-141). Because of these vehicle induced problems, patients frequently are pre-medicated with corticosteroids or histamine antagonists. Finally, because of the dosing method the full therapeutic value of the drug cannot be used. Thus, more frequent dosing would enhance systemic drug levels over time, a result that cannot be achieved with a single intravenous dose that occurs at one, two or three week intervals and is accompanied by non-linear pharmacokinetic behavior (van Tellingen O, Br. J. Cancer, 1999, 81: 330-335).

Attempts have been made to ameliorate the problems caused by the intravenous, emulsion strategy by simply giving patients the intravenous emulsion orally in the presence of cyclosporine A, a potent inhibitor of small intestinal efflux proteins (Sparreboom A, et al., Proc. Natl Acad Sci, 1997, 94: 2031-2035; Mallingre, M M et al., 2000, J Clin Onc, 2468-2475). Even though this delivery method has the potential to alleviate at least some of the problems associated with the intravenous method, the presence of Cremophor EL in the oral formulation decreases the overall absorption of paclitaxel (Bardelmeijer, H A et al., 2002, Cancer Chemother Pharmacol 49: 119-125).

Similar to this approach, the pharmaceutical industry has devised a variety of self-emulsifying drug delivery systems that package a drug like paclitaxel in a variety of lipids and surfactants that provide a dispersible matrix when the combination is ingested (Veltkamp S A et al., British J Can, 2006, 95: 729-734). Alternatively, it has been suggested that formulations that are patterned after the lipid composition of digestion phases may provide insight into better ways to solubilize water insoluble drugs (Porter C J H, et al., J Pharm Sci 93: 1110-1121, 2004). While these studies have demonstrated the importance of the digestion process as a guide or template for drug absorption, the approach is empirical requiring exhaustive studies for each drug. Moreover, this strategy is focused more on the physical chemistry of solubilization than on the biochemistry of absorption so it provides little additional insight into the molecular events that are an integral and obligatory part of the absorption process.

Another delivery strategy has been the use of liposomes as an encapsulation vehicle for a variety of drugs for different delivery routes, including oral, parenteral and transdermal (Cevc, G and Paltauf, F., eds., Phospholipids: Characterization, Metabolism, and Novel Biological Applications, pp. 67-79, 126-133, AOCS Press, Champaign, Ill., 1995). This method requires amphiphiles, compounds that have a hydrophilic or polar end group and a hydrophobic or non-polar end group, such as phospholipid, cholesterol, glycolipid or a number of food-grade emulsifiers or surfactants. When amphiphiles are added to water, they form lipid bilayer structures (liposomes) that contain an aqueous core surrounded by a hydrophobic membrane. This novel structure can deliver water insoluble drugs that are “dissolved” in its hydrophobic membrane or, alternatively, water soluble drugs can be encapsulated within its aqueous core. This strategy has been employed in a number of fields. For example, liposomes have been used as drug carriers since they are rapidly taken up by the cell and, moreover, by the addition of specific molecules to the liposomal surface they can be targeted to certain cell types or organs, an approach that is typically used for drugs that are encapsulated in the aqueous core. For cosmetic applications, phospholipids and lipid substances are dissolved in organic solvent and, with solvent removal, the resulting solid may be partially hydrated with water and oil to form a cosmetic cream or drug-containing ointment. Finally, liposomes have been found to stabilize certain food ingredients, such as omega-3 fatty acid-containing fish oils to reduce oxidation and rancidity (Haynes et al, U.S. Pat. No. 5,139,803).

Even though liposomes provide an elegant method for drug delivery, their use has been limited by cumbersome preparation methods, inherent instability of aqueous preparations and low drug loading capacity for solid, oral preparations. The utility of a dried preparation to enhance the stability and shelf life of the liposome components has long been recognized, and numerous methods have been devised to maintain the stability of liposomal preparations under drying conditions: Schneider (U.S. Pat. No. 4,229,360); Rahman et al. (U.S. Pat. No. 4,963,362); Vanlerberghe et al. (U.S. Pat. No. 4,247,411); Payne et al. (U.S. Pat. Nos. 4,744,989 and 4,830,858). The goal of all these patented methods is to produce a solid that can be re-hydrated at a later time to form liposomes that can deliver a biologically active substance to a target tissue or organ.

Surprisingly, there have been only two reports that use the dried liposome preparations themselves, with no intermediate hydration, as the delivery system. Ostlund, U.S. Pat. No. 5,932,562 teaches the preparation of solid mixes of plant sterols for the reduction of cholesterol absorption. Plant sterols or plant stanols are premixed with lecithin or other amphiphiles in organic solvent, the solvent removed and the solid added back to water and homogenized. The emulsified solution is dried and dispersed in foods or compressed into tablets or capsules. In this case, the active substance is one of the structural components of the liposome itself (plant sterol) and no additional biologically active substance was added. Manzo et al. (U.S. Pat. No. 6,083,529) teach the preparation of a stable dry powder by spray drying an emulsified mixture of lecithin, starch and an anti-inflammatory agent. When applied to the skin, the biologically active moiety is released from the powder only in the presence of moisture. Neither Ostlund nor Manzo suggest or teach the use of sterol, and lecithin and a drug active, all combined with a non-polar solvent and then processed to provide a dried drug carrying liposome of enhanced delivery rates.

Substances other than lecithin have been used as dispersing agents. Following the same steps (dissolution in organic solvent, solvent removal, homogenization in water and spray drying) as those described in U.S. Pat. No. 5,932,562, Ostlund teaches that the surfactant sodium steroyl lactylate can be used in place of lecithin (U.S. Pat. No. 6,063,776). Burruano et al. (U.S. Pat. Nos. 6,054,144 and 6,110,502) describe a method of dispersing soy sterols and stanols or their organic acid esters in the presence of a mono-functional surfactant and a poly-functional surfactant without homogenization. The particle size of the solid plant-derived compounds is first reduced by milling and then mixed with the surfactants in water. This mixture is then spray dried to produce a solid that can be readily dispersed in water. Similarly, Bruce et al. (U.S. Pat. No. 6,242,001) describe the preparation of melts that contain plant sterols/stanols and a suitable hydrocarbon. On cooling these solids can be milled and added to water to produce dispersible sterols. Importantly, none of these methods anticipate the type of delivery method described here as a means to deliver hydrophobic, biologically active compounds.

None of the previous art suggests or teaches methods to enhance the uptake of a drug(s)/sterol/amphilphile combination at a drug loading capacity that would lead to a commercially viable drug delivery system. The stability and ultimate use of liposomal preparations have been shown to depend on the ratio of lecithin to the sterol drug combination. Thus, in order to form creams and parenteral liposomal preparations, previous work focused on the preparation of dispersions containing small liposomal particles (less than 1 Mm) by maintaining a high ratio of lecithin to the other components. This prejudice was shown by the requirement that the sum of the drug and the sterol present should not exceed about 25% and preferably about 20% of the total lipid phase present. Hence, the previous art teaches a ratio of lecithin to the sum of the sterol and drug components of at least 3.0, and preferably 4.0 [Perrier et al., U.S. Pat. No. 5,202,126 (c2, line 45), Meybeck & Dumas, U.S. Pat. No. 5,290,562 (c3, line 29)]. Moreover, the purpose of this requirement was to maintain liposomal “quality,” which was achieved with a small particle size in order to enhance the stability of the dispersion for the intended uses contained therein [Perrier et al., U.S. Pat. No. 5,202,126 (c4, line 61)]. Departure from this preferred ratio produced sediment which “detracts from the stability of the liposomes” [Perrier et al., U.S. Pat. No. 5,202,126, (c5, line 10)].

In contrast, for the preparation of oral dosage forms it was shown that a superior preparation contained a ratio of the sterol drug combination to amphiphile of 0.2 to 3.0. (Spilburg, patent application Ser. No. 11/291,126, Nov. 30, 2005). This combination produces a delivery system with the following useful and novel advantages: a dispersed solution that can be dried and re-hydrated to produce a dispersion of particles that is similar to that of the dispersion from which it was derived; high drug(s) loading capacity by minimizing the amount of amphiphile in the mix; an emulsion that is stable to conventional drying methods without the addition of large amounts of stabilizers. The dried solid so manufactured can be easily compacted in a tablet and capsule to render the hydrophobic drug bioavailable on ingestion and easily deliverable in a pharmaceutical format.

Moreover, while the previous work of my earlier application focused on the delivery of drugs that were either solids or oils, this present invention extends the utility of this method to show that the method is sufficiently robust to allow for the delivery of drugs—one that provides the proposed therapeutic benefit and one that blocks the action of small intestinal efflux proteins—to provide improved bioavailability. As a result even some cancer drugs like Paclitaxel can now be delivered orally.

All of the above described liposome-related art, either deals with cholesterol lowering or with a variety of techniques used in an attempt to solubilize some hydrophobic drugs using specific lipids. None teach or suggest a generalized approach to address the two problems associated with hydrophobic, and especially xenobiotic drug uptake—lack of water dispersibility and interaction with small intestinal cell drug exporters, such as P-gp.

An object of the invention is to enhance the biological activity of a hydrophobic drug substance in an oral dosage form through the use of a combination of amphiphiles, surfactants or emulsifiers and a second drug-like substance that blocks small intestinal drug exporters, such as P-gp.

A further object is to provide new oral dosage formulations that can be used for many cancer chemotherapeutics that are naturally occurring chemically complex molecules.

A still further object is to develop a new oral dose form for Paclitaxel.

The method of accomplishing these as well as other objectives will become apparent from the detailed description.

### BRIEF DESCRIPTION OF THE DRAWINGS

FIG. 1 shows the absorption of paclitaxel in female dogs using the liquid formulation systems described in Example 1.

FIG. 2 shows the absorption of paclitaxel in female dogs using the solid formulation systems described in Example 2.

### SUMMARY OF THE INVENTION

Compositions and methods are provided herein for enhancing the bioavailability of hydrophobic, poorly water soluble compounds and drugs. The compositions contain at least four components—an emulsifier or amphiphile; a sterol (preferably plant-derived); a hydrophobic active or drug compound; and an inhibitor of the small intestinal drug efflux protein. The compositions are especially useful for cancer Chemotherapeutics.

### DETAILED DESCRIPTION OF A PREFERRED EMBODIMENT

There are at least three ways to use the delivery system of this invention. In Method I, the four ingredients are mixed together and processed to provide a single capsule dose. This is a good system but it delivers the drug and the efflux inhibitor at the same time, which may not be optimal for some cases. The second way (Method II) allows for the separate preparation of the active drug and the efflux inhibitor and then dosing them in the same capsule. This allows for each component to be prepared with a different emulsification system that allows the efflux inhibitor to be dispersed more rapidly than the active drug. And the third way (Method III) takes this one step further by preparing them separately and dosing them in separate capsules. In this way the efflux inhibitor can be dosed at any time before the active drug.

### Method I

(a) An amphiphile, such as lecithin or one of its derivatives, a sterol (preferably a plant-derived sterol), the active drug substance and an inhibitor of the drug efflux protein are mixed in a non-polar solvent (preferably ethyl acetate or heptane) at its boiling point.
(b) A solid is collected after the solvent is driven off at elevated temperature to maintain the solubility of all the components.
(c) The solid is broken into small pieces and dispersed with vigorous stirring in water at a temperature that is less than the decomposition temperature of one of the components or the boiling point of water, whichever is lower.
(d) The milky solution is passed through a Gaulin Dairy Homogenizer (or suitable equivalent) operating at maximum pressure; and thereafter
(e) The milky solution is spray dried or lyophilized to produce a solid that can be incorporated into tablets or capsules, providing the appropriate excipients are added. Optionally, a suitable drying aid is added (Maltrin, Capsule M or suitable equivalent) to assist the drying process.

### Method II

The active drug substance and an inhibitor of the drug efflux protein are prepared separately as described in Method I. The two spray dried powders are then dry blended together and delivered in a single tablet or capsule.

### Method III

The active drug substance and the inhibitor of the drug efflux protein are each prepared separately as described in Method I. The powder containing the active drug is packed into its own tablet or capsule and the powder containing the inhibitor of the drug efflux protein is packed separately into its own tablet or capsule. This method allows for the administration of the inhibitor of the drug efflux protein at various times before the administration of the active drug substance.

If the active drug substance and the inhibitor of the drug efflux protein are not compatible with organic solvents, the preparation of the water-dispersible powders can be achieved by using other manufacturing techniques such as, jet cooking, preparation of melts providing the various compounds are stable at the melting temperature of the substance used as the “solvent,” and high pressure compression and extrusion of blends of the various components.

Numerous amphiphilic emulsifiers have been described, but since this invention contemplates pharmaceutical application only those compounds that have been approved for human use are acceptable. A preferred emulsifier is lecithin derived from egg yolk, soy beans or any of its chemically modified derivatives, such as lysolecithin. Lecithin is not only an excellent emulsifier and surfactant, it also has many health benefits that are beneficial when used as the contemplated pharmaceutical formulation agent described here [Cevc, G. and Paltauf, F., eds., Phospholipids: Characterization, Metabolism, and Novel Biological Applications, pp. 208-227 AOCS Pres, Champaign, Ill., 1995]. While many grades and forms are available, de-oiled lecithin produces the most consistent results. Typical commercially available examples are Ultralec P, Ultralec F and Ultralec G (Archer Daniels Midland, Decatur, Ill.) or Solec 8160, a powdered, enzyme-modified lecithin (Solae, St. Louis, Mo.).

Other emulsifiers can be successfully used including, but not limited to mono and diglycerides, diacetyltartaric acid esters of mono and diglycerides, monoglyceride phosphate, acetylated monoglycerides, ethoxylated mono and diglycerides, lactylated monoglycerides, propylene glycol esters, polyglycerol esters, polysorbates, sorbitan esters, sodium and calcium stearoyl lactylate, succinylated monoglycerides, sucrose esters of fatty acids, fatty alcohols, sodium salts of fatty acids. In certain instances, combinations of these emulsifiers may also be used.

A variety of sterols and their ester derivatives can be added to the emulsifier(s) to enhance the aqueous dispersibility in the gut in the presence of bile salts and bile phospholipid. While cholesterol has frequently been used for this purpose, its absorption can lead to elevated LDL-cholesterol levels, making it a poor choice for the pharmaceutical applications contemplated here. Plant-derived sterols, especially those derived from soy and tall oil, are the preferred choice since they have been shown to lower LDL-cholesterol and they are considered to be safe (Jones P J H et al., Can J. Physiol Pharmacol 75: 227-235, 1996). Specifically, this invention contemplates the use of mixtures including, but not limited to sitosterol, campesterol, stigmasterol and brassicasterol and their corresponding fatty acid esters prepared as described elsewhere (Wester I., et al., “Stanol Composition and the use thereof”, WO 98/06405). The reduced forms of the above-mentioned sterols and their corresponding esters are the most preferred, since they also lower human LDL-cholesterol and their absorption is from five- to ten-fold less than that of their non-reduced counterparts (Ostlund R E et al., Am. J. of Physiol, 282: E 911-E916, 2002; Spilburg C et al., J Am Diet Assoc 103: 577-581, 2003).

Hydrophobic drugs and potential drugs may be selected from any therapeutic class including but not limited to anesthetics, anti-asthma agents, antibiotics, antidepressants, anti-diabetics, anti-epileptics, anti-fungals, anti-gout, anti-neoplastics, anti-obesity agents, anti-protozoals, anti-phyretics, anti-virals, anti-psychotics, calcium regulating agents, cardiovascular agents, corticosteroids, diuretics, dopaminergic agents, gastrointestinal agents, hormones (peptide and non-peptide), immunosuppressants, lipid regulating agents, phytoestrogens, prostaglandins, relaxants and stimulants, vitamins/nutritionals, xanthines and xenobiotics. A number of criteria can be used to determine appropriate candidates for this formulation system, including but not limited to the following: drugs or organic compounds that are known to be poorly dispersible in water, leading to long dissolution times or; drugs or organic compounds that are known to produce a variable biological response from dose to dose or; drugs that are oils that are difficult to deliver in a conventional tablet or capsule delivery system or; drugs or organic compounds that have been shown to be preferentially soluble in hydrophobic solvent as evidenced by their partition coefficient in the octanol water system or; drugs that are preferentially absorbed when consumed with a fatty meal or; drugs that can only be delivered intravenously or by injection. In addition to these components, other ingredients may be added that provide beneficial properties to the final product, such as vitamin E to maintain stability of the active species.

Inhibitors of the small intestinal efflux protein or of cytochrome P450 include, but are not limited to, verapamil, cyclosporin A, cyclosporine D, erythromycin, quinine, fluphenazine, reserpine, progesterone, tamoxifen, mitotane, annamycin, biricodar, elacridar, tariquidar and zosuquidar.

For those drugs that are compatible with organic solvents, all the formulation components are dissolved in a suitable non-polar organic solvent, such as chloroform, dichloromethane, ethyl acetate, pentane, hexane, heptane or supercritical carbon dioxide. The choice of solvent is dictated by the solubility of the components and the stability of the drug at the temperature of the solvent. The preferred solvents are non-chlorinated and for heat stable compounds, heptane is the most preferred solvent because of its high boiling point, which increases the overall solubility of all the components.

The weight fraction of each component in the final four-component mixture depends on the nature of the hydrophobic compound(s), the nature of the emulsifier amphiphile used to prepare the blend and the intended use of the final product—tablet, capsule, food product or beverage. Regardless of method, the goal is to produce an emulsified mixture of drug, inhibitor of the efflux protein, sterols and amphiphile so that the amount of amphiphile in the system is minimized relative to the other components. To achieve this end for Method I, in the total blend containing all four components, the weight fraction of each component is given in the table below.

FRACTION BY WEIGHT OF EACH COMPONENT
IN THE FINAL BLEND

| Component | Broad Range | Preferred Range |
|---|---|---|
| Amphiphile (emulsifier) | 0.075-0.95 | 0.20-0.80 |
| Sterol | 0.02-0.75 | 0.10-0.60 |
| Drug active effective amt. | 0.02-0.50 | 0.10-0.40 |
| Intestinal efflux inhibitor | 0.012-0.50 | 0.10-0.40 |

The ranges described in the table above also apply for Methods II and III. However, for these methods the active drug and the inhibitor of the efflux protein are prepared separately, but when they are combined together in the same capsule or in separate capsules, the ranges above still apply. Importantly, in all methods, sufficient amphiphile must be present to allow dispersibility.

After all the components are dissolved at the desired ratio in the appropriate solvent, the liquid is removed at elevated temperature to maintain the solubility and stability of all the components. Residual solvent can be removed by pumping under vacuum. Alternatively, the solvent can be removed by atomization as described in U.S. Pat. Nos. 4,508,703 and 4,621,023. The solid is then added to water at a temperature that is less than the decomposition temperature of one of the components or the boiling point of water, whichever is lower. The mixture is vigorously mixed in a suitable mixer to form a milky solution, which is then homogenized, preferably with a sonicator, Gaulin dairy homogenizer or a microfluidizer. The water is then removed by spray drying, lyophilization or some other suitable drying method. Before drying, it is helpful but not necessary, to add maltrin, starch, silicon dioxide, calcium silicate or sodium croscarmellose to produce a flowable powder that has more desirable properties for filling capsules, compression into tablets or addition to certain medical foods. The addition of a suitable antacid, such as calcium carbonate or the like, to the powder at a weight percent of 0.5 to 10.0 stabilizes and/or activates the components in the blend to produce a superior product. For some blends, either wet or solid granulation produces a superior solid with a greater bulk density.

The dried liposomal blend described above is the starting point for a variety of flexible delivery systems described below. Since the key components of the powdered formulation system are compounds that are an integral result of the digestive process, they are compatible with food delivery systems that can be especially designed for children and the elderly. The powdered drug/plant sterol/lecithin blend described above can be easily dispersed in milk or other beverages for convenient delivery to neonates and infants. Moreover, the absence of pancreatic lipolytic activity and low concentrations of bile salt are not an impediment to drug absorption since the drug is packaged in a system that contains components that are the end product of the digestive process. This is of special importance for neonates and adults with pancreatic insufficiency, such as cystic fibrosis patients. In summary, the proposed formulation system provides a seamless transition from neonates—powder dispersed in milk—to children—powder compressed in a chewable tablet—to adults—powder compressed in a conventional tablet or capsules—to the elderly—powder dispersed in beverages or other supplemented drinks.

There are other known methods that can be used to prepare tablets. After the components have been mixed at the appropriate ratio in organic solvent, the solvent can be removed as described above. The solid material so prepared can then be compressed at elevated pressure and extruded into a rope. The rope can be cut in segments to form tablets. This method is similar to that described in U.S. Pat. No. 6,312,703, but the inventor did not recognize the importance of pre-mixing the components in organic solvent. While this previous method produces a tablet, the components may not be as freely dispersible in bile salt and phospholipid when they are not pre-mixed in organic solvent. Alternatively, the solid material that results from homogenization and spray drying can be compressed at high pressure and extruded to form a rope that can be cut into tablets.

The precise details of tableting technique are not a part of this invention, and since they are well-known they need not be described herein in detail. Generally pharmaceutical carriers which are liquid or solid may be used. The preferred liquid carrier is water, but milk can also be used especially for neonates and infants. Flavoring material may be included in the solutions as desired.

Solid pharmaceutical carriers such as starch, sugar, talc, mannitol and the like may be used to form powders. Mannitol is the preferred solid carrier. The powders may be used as such for direct administration to a patient, or instead, the powders may be added to suitable foods and liquids, including water, to facilitate administration.

The powders also may be used to make tablets, or to fill gelatin capsules. Suitable lubricants like magnesium stearate, binders such as gelatin, and disintegrating agents like sodium carbonate either alone or in combination with citric acid may be used to form the tablets.

While not precisely knowing why, and not wishing to be bound by any theory of operability, the fact is that for difficulty soluble drugs this composition and combination of steps achieved higher absorption and lower variability of absorption.

In the examples to follow, the novelty and utility of the method will be shown in both liquid and solid delivery systems. The improvement in the uptake will be shown by comparing the formulation system to that available in the corresponding commercially available unformulated drug. To these ends, pharmacokinetic studies were performed in five naïve, female beagle dogs with each drug dosed in a formulation system using a crossover design, with a one week wash out period between doses. All animal work was performed following procedures for animal care and housing that were in accordance with the Guide for the Care and Use of Laboratory Animals (Institute of Laboratory Animal Resources, Commission on Life Sciences, National Research Council, National Academic Press, 1996). Following a 16-hour fast, the animals were fed a small amount (approximately ¼ can) of Hill/s Science Diet A/D and thirty minutes later each animal was orally dosed with one of the formulations of the appropriate test article. Blood samples were drawn 0.5, 1.0, 1.5, 3.0, 4.5, 8.0, and 24 hours after dosing.

### Example 1

Liquid Preparations—Paclitaxel. Solid Paclitaxel (20 mg), plant sterols (20 mg) and lysolecithin (60 mg) were added to each of five plastic tubes and chloroform was added (1.0 mL) to each sample tube. The solvent was removed under a stream of nitrogen with gentle warming in a 60° C. water bath and then pumped on to remove residual solvent. On the day of the experiment, water (10.0 mL) was added and the mixture was sonicated for 30 seconds at 50% power with a Branson Digital Sonifier, equipped with a ⅛″ tapered tip. The liquid was then dosed to the animal with a syringe. Water was then added to the syringe and the washing was administered to the dog.

Liquid Preparations—Paclitaxel+Cyclosporin A (P-gp Inhibitor. Paclitaxel was processed as above except 5.0 mL of water was added before sonication.

Solid cyclosporin A (80 mg), plant sterols (80 mg) and lecithin (160 mg) were added to each of five plastic tubes and chloroform was added (1.0 mL) to each sample tube. The P-gp inhibitor was processed as described above for Paclitaxel except 5.0 mL of water was added for sonication. After sonication, the Paclitaxel solution and cyclosporin solution were mixed together and the milk-like combination was delivered in a syringe to a dog on the day of the experiment.

Control Experiment—Solid Unformulated Paclitaxel. Calcium carbonate (50 mg), Maltrin® (75 mg) and silicon dioxide (3 mg) were weighed and added to a “000” gelatin capsule. Separately, Paclitaxel (20 mg) was weighed and added to the other ingredients in the capsule. The capsule cap was installed to the bottom piece and the contents were vigorously shaken to blend the solids.

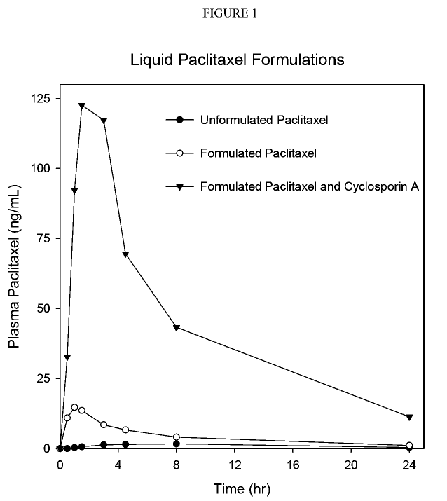

Absorption Experiments With Liquid Formulations. After dosing with each formulation, all the blood samples were collected in a sodium heparin anticoagulant tube, processed to plasma and frozen at −80° C. Plasma Paclitaxel concentration at each time point for each of the five dogs was determined by high throughput liquid chromatographic-tandem mass spectrometric quantification at Bioanalytical Systems (McMinnville, Oreg.). As shown in FIG. 1, there is a marked increase in Paclitaxel absorption for this Cremophor E-free liquid formulation when compared to that for the unformulated Paclitaxel. To quantitate the absorption changes, the area under the curve (AUC_{0÷∞}) was calculated for each formulation system and the results are shown in the Table below. Compared to the unformulated Paclitaxel, there was a 4.1-fold increase in absorption from the formulation system alone (p=0.18), and a statistically significant (p=0.008) 41-fold increase when compared to the formulated Paclitaxel cyclosporin A combination.

EFFECT OF LIQUID FORMULATION ON PACLITAXEL UPTAKE

| Formulation | AUC_{0-∞} (ng/mL h⁻¹) |
|---|---|
| (A) Unformulated Paclitaxel (A) | 28.9 ± 7.1 |
| (B) Formulated Paclitaxel (B) | 118.1 ± 57.6 |
| (C) Formulated Paclitaxel plus Cyclosporin A | 1,189 ± 239.2 |

A vs B, p = 0.18,
A vs C, p = 0.008;
B vs C, p = 0.008

These data indicate that an aqueous formulation containing plant sterols and an amphiphile like lysolecithin provide a matrix that enhances the absorption of Paclitaxel without the need of Cremophor E and alcohol. Importantly, the formulation system was well tolerated by all the animals.

### Example 2

A solid formulation method was also used to determine the effect of the formulation system in the presence or absence of cyclosporin A (P-gp inhibitor).

Solid Preparation—Paclitaxel. Solid Paclitaxel (300 mg), soy sterols (300 mg) and lysolecithin (900 mg) were added to a 30 mL glass tube and chloroform (3.0 ml) was added. After the solids were dissolved with gentle heating in a 60° C. water bath, the solvent was removed under a stream of nitrogen. The mass was then pumped on under vacuum to remove residual solvent. Addition of water (15 mL) softened the solid mass and the mixture was then sonicated in an ice bath for two minutes on 40% power, followed by two minutes sonication on 50% power and then two minutes sonication on 60% power. The milky solution was then transferred to a lyophilization jar and croscarmellose and fumed silica were added followed by an additional two-minute period of sonication at 60% power to disperse the solids. The milky solution was then shell frozen in a dry ice-acetone bath and lyophilized. Lyophilized formulated Paclitaxel (110 mg, 21 mg Paclitaxel) was dry granulated with calcium carbonate, Maltrin and silicon dioxide. There was a noticeable decrease in the bulk density and the flowable powder was packed into a “000” capsule. This granulation process was repeated five times for five separate capsules.

Solid Preparations—Formulated Paclitaxel+Cyclosporin A. Solid cyclosporin A (500 mg), soy sterols (500 mg) and lecithin (1000 mg) were added to each of two 30 mL glass tubes and chloroform (3.0 ml) was added. A lyophilized blend of the components was prepared as described above for solid Paclitaxel. To increase the bulk density of the cyclosporin blend, the powder was wet granulated with calcium carbonate by spraying with 10% polyvinylpyrrolidone dissolved in 91% isopropanol. The blend was set aside to air dry for 48 hours and the pale yellow solid was collected and passed through a #10 screen. Larger granules were milled in a coffee grinder and the solid was re-screened. Capsules were filled in two steps. First, cyclosporin granules were weighed into a “000” capsule and allowed to stand in an upright position with the cap not installed. Second, dry granulated Paclitaxel was then added, and the capsule head was firmly installed.

Control Experiment—Solid Unformulated Paclitaxel. Calcium carbonate (50 mg), Maltrin® (75 mg) and silicon dioxide (3 mg) were weighed and added to a “000” gelatin capsule. In a separate weighing, Paclitaxel (20 mg) was weighed and added to the other ingredients in the capsule. The capsule cap was installed to the bottom piece and the contents were vigorously shaken to blend the solids.

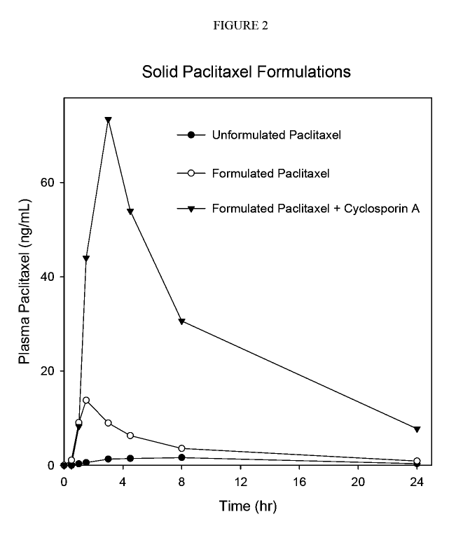

Absorption Experiment With Solid Formulations. After dosing with each formulation, all the blood samples were processed and analyzed as described for the liquid formulations. As shown in FIG. 2, there is a marked increase in Paclitaxel absorption for the two solid formulations when compared to that for the unformulated Paclitaxel. To quantitate the absorption changes, the area under the curve (AUC_{0→∞}) was calculated for each formulation system and the results are shown in the Table below. Compared to the unformulated Paclitaxel, there was a statistically significant 3.5-fold (p=0.02) increase in absorption from the formulation system alone, and a 26-fold (p=0.008) increase when compared to the formulated Paclitaxel cyclosporin A combination.

EFFECT OF SOLID FORMULATION ON PACLITAXEL UPTAKE

| Formulation | AUC_{0-∞} (ng/mL h⁻¹) |
|---|---|
| (A) Unformulated Paclitaxel | 28.9 ± 7.1 |
| (B) Formulated Paclitaxel | 101.2 ± 23.4 |
| (C) Formulated Paclitaxel plus Cyclosporin A | 752.1 ± 134.5 |

A vs B, p = 0.02,
A vs C, p = 0.005;
B vs C, p = 0.008

Taken together, these two experiments indicate that improved paclataxel absorption occurs when the xenobiotic is formulated in a sterol emulsifier combination, which is designed to enhance its dispersibility in the small intestinal lumen. Even though this produces an impressive 3.5-4.0-fold increase in absorption when compared to that of the unformulated solid, the small intestinal efflux transporter expels much of the absorbed drug. The addition of an inhibitor of the export protein (cyclosporine A), formulated in the same system as that used for Paclitaxel, increases the absorption 25-40-fold relative to that for the unformulated solid, demonstrating that optimum absorption occurs when the exporter is inhibited and when the hydrophobic components are in a dispersible formulation. To my knowledge, this is the first demonstration that Paclitaxel can be efficiently absorbed as a solid.

The above described examples are illustrative of the invention, which is of course broader than the specific examples. The scope of the invention is defined by the appended claims.

What is claimed is:

1. A drug delivery composition for normally difficultly soluble hydrophobic crystalline drug actives, comprising:
an emulsifier, a plant derived sterol (stanol) or ester derived from the sterol (stanol);
a drug active effective amount of a hydrophobic drug; and
a small but inhibiting effective amount of an inhibitor of small intestine efflux proteins.

2. The composition of claim 1 wherein the emulsifier is one which is approved for food or pharmaceutical use.

3. The composition of claim 2 wherein the emulsifier is selected from the group consisting of lecithin, lysolecithin, mono or diglyceride, diacetyltartaric acid esters of mono and diglycerides, monoglyceride phosphate, acetylated monoglycerides, ethoxylated mono and diglycerides, lactylated monoglycerides, propylene glycol esters, polyglycerol esters, polysorbates, sorbitan esters, sodium and calcium stearoyl lactylate, succinylated monoglycerides, sucrose esters of fatty acids, fatty alcohols, sodium salts of fatty acids, tween or combinations thereof.

4. The drug delivery composition of claim 1 wherein the plant derived sterol (stanol) or plant derived sterol (stanol) ester is derived from a vegetable or tall oil source.

5. The composition of claim 1 wherein the emulsifier is from about 7.5% by weight to about 95% by weight of the composition; the sterol from about 2% by weight to about 75% by weight of the composition; the drug active from about 2% to about 50% by weight of the composition; and, the intestine efflux inhibitor from about 2% to 50% by weight of the total composition.

6. The composition of claim 5 wherein the emulsifier is from about 20% by weight to about 80% by weight of the composition; the sterol from about 10% by weight to about 60% by weight of the composition; the drug active from about 10% to about 40% by weight of the composition; and, the intestine efflux inhibitor from about 10% to 40% by weight of the total composition.

7. The composition of claim 1 wherein the drug delivery composition includes as an additional hydrophobic compound, vitamin E.

8. The composition of claim 1 wherein the drug active is selected from the group consisting of anesthetics, anti-asthma agents, antibiotics, antidepressants, anti-diabetics, anti-epileptics, anti-fungals, anti-gout, anti-neoplastics, anti-obesity agents, anti-protozoals, anti-phyretics, anti-virals, anti-psychotics, calcium regulating agents, cardiovascular agents, corticosteroids, diuretics, dopaminergic agents, gastrointestinal agents, hormones (peptide and non-peptide), immunosuppressants, lipid regulating agents, phytoestrogens, prostaglandins, relaxants and stimulants, vitamins/nutritionals, xanthines and xenobiotics.

9. The composition of claim 8 wherein the drug active is a xenobiotic.

10. The composition of claim 9 wherein the xenobiotic is selected from the group consisting of Taxanes, Camptothecins, Anthrocyclins, Vinca Alkaloids and Epipodophyllotoxins.

11. The Composition of claim 9 wherein the Xenobiotic is Paclitaxel.

12. The composition of claim 9 wherein the Xenobiotic is Topetocan.

13. The composition of claim 9 wherein the Xenobiotic is Doxorubicin.

14. The composition of claim 9 wherein the Xenobiotic is Vinblastine.

15. The composition of claim 9 wherein the Xenobiotic is Etoposide.

16. The composition of claim 1 in which all of the compositions is provided in a typical single pharmaceutical oral dose delivery system.

17. The composition of claim 1 wherein the small intestine efflux inhibitor is selected from the group consisting of verapamil, cyclosporin A, cyclosporine D, erythromycin, quinine, fluphenazine, reserpine, progesterone, tamoxifen, mitotane, annamycin, biricodar, elacridar, tariquidar and zosuquidar.

18. The composition of claim 1 wherein the drug active and small intestine efflux inhibitor are separately mixed with the sterol and emulsifier, before mixing with each other to provide the drug delivery composition.

19. The composition of claim 1 which is packaged as two oral doses, one containing drug active, sterol and emulsifier, and the other containing sterol, emulsifier and small intestine efflux inhibitor.

20. The composition of claim 1 in which the drug active sterol and emulsifier are dried to a powder and then blended with sterol, emulsifier and small intestine efflux inhibitor which has been dried to a powder.

21. The composition of claim 1 which is an oral dosage selected from a tablet and a capsule.

22. The composition of claim 1 wherein the oral dosage composition is combined with a beverage or medical food product.

23. A tablet formed from the composition of claim 1 by subjecting the material to compression or extrusion for at least 15 seconds at a pressure of at least 100 psig.

24. The method of preparing a drug delivery system for normally difficultly soluble hydrophobic compounds, comprising:
mixing together with a non-polar solvent an emulsifier(s) or mixtures thereof; a plant derived sterol (stanol) or esters derived from plant sterol (stanol) in which the fatty acid ester moiety is derived from a vegetable or tall oil; a drug active; and
an inhibitor of the small intestinal drug efflux protein;
removing the solvent to leave a solid residue of the mixed components;
adding water to the solid residue of the mixed components at a temperature less than the decomposition temperature of any one of the mixed components;
homogenizing the aqueous mixture;
drying the homogenized mixture; and
providing the dried solid residue of the mixed components in a solid pharmaceutical carrier format.

25. The method of claim 24 wherein the non-polar organic solvent is selected from the group consisting of ethyl acetate, chloroform, dichloromethane, isopropanol, carbon dioxide and heptane.

26. The method of claim 24 wherein the non-polar organic solvent is at its boiling point.

27. The method of claim 24 wherein the non-polar organic solvent is removed by elevating the temperature above the solvent's boiling point.

28. The method of claim 24 wherein the dried solid residue of the mixed components is dispersed in water with vigorous stirring at a temperature less than the decomposition temperature of any of the mixed components.

29. The method of claim 24 wherein an additional step, prior to final drying includes homogenizing of the water dispersed mixed components.

30. The method of claim 24 wherein the solid formed after solvent removal is pulverized in an appropriate mill, grinder or processor to produce a dispersible powder.

31. The method of claim 24 wherein the solvent removal continues until a solid residue that contains less than 0.5% solvent is provided.

32. The method of claim 24 wherein the powder from claim 29 is added with vigorous stirring to water at a temperature that is less than the decomposition temperature of any of the mixed components.

33. The method of claim 24 wherein water is introduced directly to the un-pulverized dried solid residue.

34. The method of claim 33 wherein the water is at a temperature that is less than the decomposition temperature of any one of the mixed components.

35. The method of claim 24 wherein the aqueous mixture is homogenized in a homogenizer selected from the group consisting of a Gaulin homogenizer, a French press, a sonicator, and a microfludizer.

36. The method of claim 24 wherein the homogenized aqueous mixture is dried in a drier selected from the group consisting of spray driers and lyophilizers.

37. The method of claim 36, wherein a drying aid selected from the group consisting of starch, silicon dioxide and calcium silicate is added.

38. The method of claim 37 wherein a suitable antacid such as calcium carbonate is blended with the dried powder.

39. The method of claim 37, wherein the antacid is added between 0.1% and 10% by weight.

40. The method of claim 39 wherein the antacid is added at 3.5% by weight.
